(19) United States
(12) Patent Application Publication
(10) Pub. No.: US 2013/0274766 A1
(43) Pub. Date: Oct. 17, 2013

(54) MEDICAL DEVICE WITH EXTERNAL ACTUATION LINE

(71) Applicant: BOSTON SCIENTIFIC SCIMED, INC., Maple Grove, MN (US)

(72) Inventors: Brad M. Isaacson, Lancaster, MA (US); Oscar R. Carrillo, JR., Attleboro, MA (US); Jason Romano, Wakefield, RI (US)

(73) Assignee: BOSTON SCIENTIFIC SCIMED, INC., Maple Grove, MN (US)

(21) Appl. No.: 13/791,303

(22) Filed: Mar. 8, 2013

Related U.S. Application Data
(60) Provisional application No. 61/624,713, filed on Apr. 16, 2012.

(51) Int. Cl. A61B 17/12; A61B 17/00

(52) U.S. Cl. CPC A61B 17/12013; A61B 17/00
USPC 606/140; 606/1; 606/139; 606/142; 606/144; 29/428

(57) ABSTRACT

An actuation line is positioned external to the support structure lumen of a medical device. The medical device, which may be a ligation banding cap assembly, may comprise a support structure having a support structure lumen therethrough, an external channel on the outer surface of the support structure, and an actuation line positioned at least in part in the external channel. The external channel may be defined by raised ridges on either side of the external channel. The external channel may be recessed in the outer surface of the support structure. When the medical device is attached to a second elongated medical device, the actuation line is external to the support structure lumen of the medical device and the second elongated medical device.

The present application claims priority to U.S. provisional application Ser. No. 61/624,713 filed Apr. 16, 2012, the disclosure of which is incorporated herein by reference in its entirety.

### TECHNICAL FIELD

The invention relates to medical devices having actuation lines. In some embodiments, the invention relates to a ligation banding cap assembly having one or more actuation lines.

### BACKGROUND OF THE INVENTION

Various medical procedures involve medical devices having actuation lines in the form of trigger wires or sutures. For example, certain ligation banding cap assemblies use actuation lines for the deployment of one or more ligation bands.

Ligation is a medical procedure whereby a physician ties off or mechanically constricts a piece of body tissue with a ligature such as a suture, clip or band. In certain procedures, the purpose of ligation is to impede or obstruct the flow of blood, fluids and/or other bodily materials through the tissue. For example, a physician can remove target tissue by ligating it to obstruct circulation through the target tissue, thereby causing the tissue to die and slough off. The purpose of ligation may also be to hold tissue to be cauterized or resected, as in an endoscopic mucosal resection (EMR) procedure.

For ligating tissue inside a body cavity, orifice, or lumen, physicians often use an endoscope to access the target tissue and ligate it. In one such form of endoscopic ligation, the physician attaches a ligation banding cap to the end of an endoscope. One or a plurality of ligation bands are stretched around the ligation banding cap and can be deployed by the physician. The physician uses the endoscope to position each stretched ligation band over the target tissue and then releases the band onto the tissue so that the band contracts and catches the tissue. The inward pressure of the ligation band constricts the target tissue.

Ligating instruments have been the subject of a number of patents and patent applications, including U.S. Pat. No. 5,259,366 to Reydel et al.; U.S. Pat. No. 5,269,789 to Chin, et al.; U.S. Pat. No. 5,356,416 to Chu, et al.; U.S. Pat. No. 5,398,844 to Zaslaysky, et al.; U.S. Pat. No. 5,853,416 to Tolkoff; U.S. Pat. No. 5,857,585 to Tolkoff, et al.; U.S. Pat. No. 5,913,865 to Fortier, et al.; U.S. Pat. No. 5,968,056 to Chu, et al., U.S. Pat. No. 6,235,040 to Chu, et al.; U.S. Pat. No. RE 36,629 to Zaslaysky, et al., and U.S. Patent Application Publication No. 2008/0091218 to Richardson. The disclosures of these prior U.S. patents and patent application are expressly incorporated herein by reference.

In certain previous ligating instruments, the ligation banding cap has one or more ligation bands around its outer surface. One or more actuation lines in the form of a trigger wire or suture extend from a proximal actuator located outside of the patient's body to the ligation bands. The physician can deploy the bands by an actuator which pulls the actuation line to deploy the bands.

In certain prior instruments of this type, the actuation lines are typically threaded through the support structure lumen of the ligation banding cap and through a channel of the endoscope. The physician uses the endoscope to visualize the procedure and target site. However, with this positioning of the actuation lines in the prior art devices, the actuation lines can cut into or cross the field of view, thereby obstructing the view. In addition, in some procedures it is desired to deploy one or more other instruments such as snares through the endoscope. The actuation lines can interfere with the deployment and/or use of such other instruments. Moreover, in certain instances the actuation lines can interfere with the tissue and/or inadvertently catch onto anatomical features. Accordingly, there is a need for improvement in medical devices using actuation lines.

### SUMMARY OF THE INVENTION

The present disclosure relates to positioning an actuation line external to an inner channel or lumen of a medical device.

In some embodiments, a medical device having an actuation line is provided, the medical device comprising a support structure having a support structure lumen therethrough, an external channel on the outer surface of the support structure, and an actuation line positioned at least in part in the external channel. The medical device may comprise a first raised ridge on one side of the external channel and a second raised ridge on the other side of the external channel. The external channel may be in the form of a recess in the outer surface of the support structure.

In other embodiments, a ligation banding cap assembly is provided, the ligation banding cap assembly comprising a support structure having a support structure lumen therethrough, one or more ligation bands positioned around the outer surface of the support structure, an external channel on the outer surface of the support structure, and an actuation line positioned at least in part in the external channel. The actuation line may be in communication with the one or more ligation bands such that actuation of the actuation line deploys the one or more ligation bands. The ligation banding cap assembly may be adapted to be attached to the distal end of an elongated medical device, such as an endoscope. The ligation banding cap assembly may comprise a first raised ridge on one side of the external channel and a second raised ridge on the other side of the external channel. The external channel may be in the form of a recess in the outer surface of the support structure.

The support structure may comprise a plurality of slots in a distal end, and the actuation line may extend through the external channel, through at least two slots, and back along the outer surface of the support structure to the one or more ligation bands. The actuation line may comprise one or more enlarged structures for advancing the one or more ligation bands.

In other embodiments, a method of assembling a ligation banding cap assembly to an elongated medical device is provided, the method comprising positioning the ligation banding cap assembly relative to the elongated medical device and attaching the ligation banding cap assembly to the elongated medical device, with the actuation line external to the support structure lumen of the ligation banding cap assembly and external to the elongated medical device. The elongated medical device may be an endoscope.

### BRIEF DESCRIPTION OF THE DRAWINGS

FIG. 1 shows a ligation banding cap assembly comprising an external channel.

FIG. 2 shows the ligation banding cap assembly of FIG. 1, with an actuation line positioned in the external channel.

FIG. 3 shows the ligation banding cap assembly of FIG. 2, with a ligation band positioned around the support structure of the ligation banding cap assembly.

### DETAILED DESCRIPTION

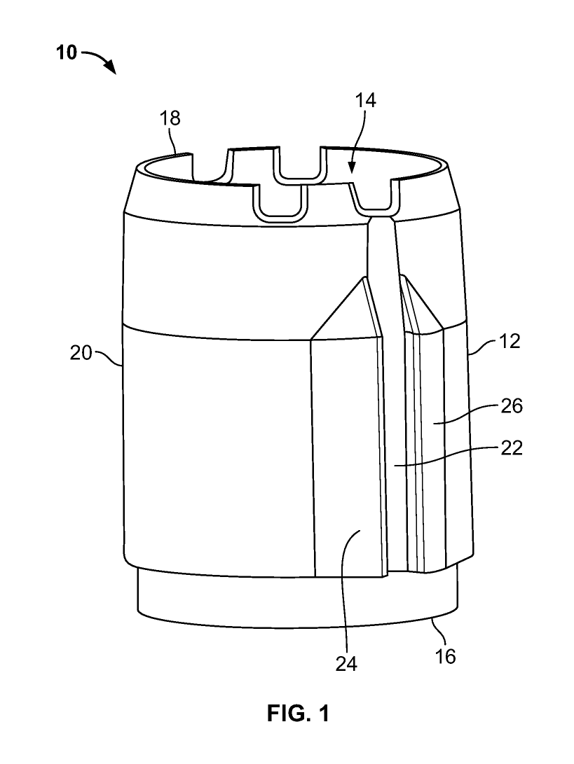

FIG. 1 shows a perspective view of a ligation banding cap assembly 10. The ligation banding cap assembly 10 comprises a support structure 12 having a support structure lumen 14 therethrough. In this example, the support structure 12 is generally cylindrical in shape and tapers to a smaller diameter at its distal end 18. At its proximal end 16, the support structure is configured to be mounted on and secured to the distal end of an endoscope or other elongated medical device.

The support structure 12 may be made of similar materials to other known ligation band support structures. For example, the support structure 12 may be made of a polycarbonate material and may be transparent to facilitate visualization through the support structure 12. Any suitable material capable of supporting the ligation bands may be used.

The support structure 12 has an outer surface 20 on which one or more ligation bands may be positioned, as discussed further below. A first external channel 22 is located on the outer surface 20 of the support structure 12. An optional second external channel (not shown) is located on an opposite side of the support structure 12 from the first external channel 22. Any suitable number of such channels may be used.

The external channel 22 may be defined by a first raised ridge 24 on one side of the external channel 22 and a second raised ridge 26 on the other side of the external channel 22. Alternatively or additionally, the external channel 22 may be in the form of a recess in the outer surface 20 of the support structure 22. In the embodiment shown in FIG. 1, the external channel is both in the form of a recess in the outer surface 20 of the support structure 22 and defined by a first raised ridge 24 on one side of the external channel 22 and a second raised ridge 26 on the other side of the external channel 22. The second external channel or other external channels may be the same as or different from the external channel 22.

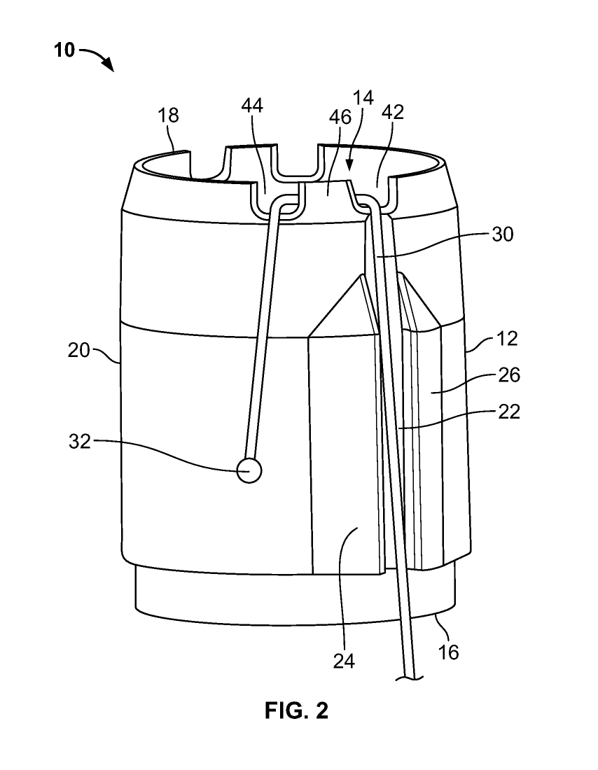

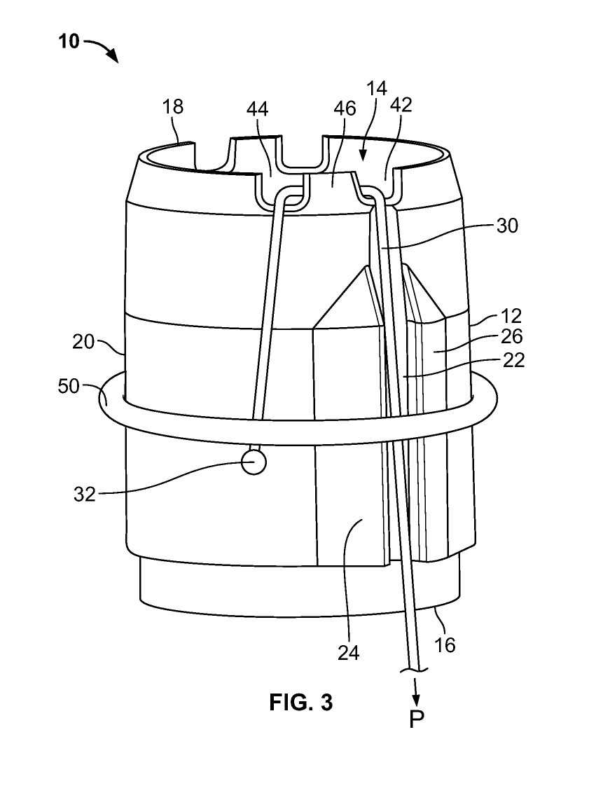

FIG. 2 shows a perspective view of the ligation banding cap assembly 10 of FIG. 1 with an actuation line 30. The actuation line 30 is positioned at least in part in the external channel 22. As described in reference to FIG. 3, the actuation line 30 is configured to be in communication with one or more ligation bands such that actuation of the actuation line 30 deploys the one or more ligation bands. The cross-section of the actuation line may take any useful size and shape, including, but not limited to, circular, semi-circular, rectangular, triangular, etc. An actuation line having a smaller cross-sectional profile will require a shallower external channel.

The support structure 12 comprises a plurality of slots 42, 44 in its distal end 18. The actuation line 30 extends through the external channel 22, through the first slot 42, around a tooth 46 defined by the two slots 42, 44, through the second slot 44, and back along the outer surface 20 of the support structure 12. Any number of slots and/or teeth may be used. The tooth shape may be in any suitable form and, in particular, the edges of the tooth should not hinder the actuation line. For example, the edges of the tooth may be squared off or rounded.

The actuation line 30 may comprise one or more ligation band contact point(s) 32 for advancing the one or more ligation bands. For example, the actuation line 30 may comprise one or more enlarged beads, knots, hooks, bars, etc., to serve as the ligation band contact point(s) 32.

Additional actuation lines may be provided. For example, in the illustrated embodiment, a second external channel (not shown) is provided opposite the external channel 22. A second actuation line may be positioned in the second external channel in a similar manner to the way the first actuation line 30 is positioned in the first external channel 22. Furthermore, more than one actuation line may lie in a channel 22 as long as its location does not negatively affect the use of another actuation line.

FIG. 3 shows a perspective view of the ligation banding cap assembly 10 of FIG. 2 with a ligation band 50 positioned around the support structure 12. A plurality of ligation bands 50 may be positioned around the support structure 12 in a manner as is known in the art, for potential sequential deployment.

As shown in FIG. 3, after the actuation line 30 wraps around the tooth 46, it extends back along the outer surface 20 of the support structure 12 to the ligation band 50. The ligation band contact point 32 is positioned behind the ligation band 50. In this manner, when the actuation line is pulled in the direction P, the ligation band contact point 32 can advance the ligation band 50 so that it advances toward the distal end 18 of the support structure 12 and deploys off of the distal end 18 of the support structure 12. The actuation line 30 may advance the ligation band(s) 50 in alternative ways. For example, the actuation line 30 may be wrapped around the ligation band(s) 50 in a manner as is known in the art to advance the ligation band(s) 50.

The raised ridges 24, 26 serve to raise the ligation band(s) 50 above the external channel 22. With this arrangement, the ligation band(s) 50 do not interfere with the actuation line 30 and vice versa. Additionally or alternatively, the external channel 22 may be sufficiently recessed into the outer surface 20 of the support structure 12 such that the raised ridges 24, 26 are not necessary. The external channel may take any suitable form, including a wide slot or a narrow slot, as long as its shape and size does not negatively affect the use of the actuation line(s).

The raised ridges, such as raised ridges 24, 26, may be provided by any number of means. For example, they may be integral components of the support structure 12, which may be provided by a manufacturing method known in the art including, but not limited to, machining and molding. The raised ridges also may be formed separately and attached during manufacturing. In some embodiments, the ridges may be provided in or on a sheath which is attached to the support structure 12. In such embodiments, the raised ridges may be formed, for example, as part of the sheath wall or by folding the wall to create ridges.

The ligation banding cap assembly 10 of FIG. 3 is adapted to be mounted on and secured to the distal end of an elongated medical device, such as an endoscope, guide tube, resection device such as an EMR bander or ESD device, catheter or other device, in a manner as is known in the art. When assembled, the actuation line 30 is external to the elongated medical device or endoscope. A jacket or casing may be provided for the actuation line 30 to pass through from the distal end of the elongated medical device or endoscope to the proximal end. The jacket or casing may be secured to the external surface of the elongated medical device or endoscope along its entire length or at certain places along its length. The proximal end of the actuation line 30 may be attached to a suitable actuator, as is known in the art. The actuator allows the operator to pull the actuation line in the direction P.

It will be appreciated that when a ligation banding cap assembly as shown and described herein is attached to the distal end of an elongated medical device, the actuation line(s) can be completely external to the support structure lumen of the ligation banding cap assembly and external to the elongated medical device. In this manner, the actuation line(s) cannot cut into or cross the field of view. In addition, the actuation line(s) cannot interfere with the deployment and/or use of instruments or tissue in the support structure lumen.

Embodiments other than those illustrated are of course possible. While a ligation banding cap assembly has been illustrated, other medical devices with one or more actuation lines can benefit from an external channel as described herein. For example, the deployment device is not limited to deploying ligation bands but alternatively may be a deployment device for deploying one or more fasteners, clips, staples, sutures, fiducial markers, etc.

The foregoing embodiments are merely examples of embodiments within the scope of the invention. Other embodiments are possible that incorporate one or more of the features and/or advantages of the above-described embodiments. This invention thus embraces other embodiments within the scope of the claims.

What is claimed is:

1. A medical device having an actuation line, the medical device comprising:
a support structure having a support structure lumen therethrough, the support structure having an outer surface;
an external channel on the outer surface of the support structure; and
an actuation line positioned at least in part in the external channel.

2. The medical device of claim 1, wherein the medical device further comprises at least one deployment device, wherein the actuation line is in communication with the deployment device such that actuation of the actuation line deploys the deployment device.

3. The medical device of claim 2, wherein the deployment device comprises a ligation device.

4. The medical device of claim 2, wherein the deployment device comprises a band, clip, staple or suture.

5. The medical device of claim 1, wherein the medical device is adapted to be attached to the distal end of an elongated second medical device.

6. The medical device of claim 5, wherein the elongated second medical device is an endoscope.

7. The medical device of claim 1, wherein the medical device comprises a first raised ridge on one side of the external channel and a second raised ridge on the other side of the external channel.

8. The medical device of claim 1, wherein the external channel is in the form of a recess in the outer surface of the support structure.

9. A ligation banding cap assembly comprising:
a support structure having a support structure lumen therethrough, the support structure having an outer surface;
one or more ligation bands positioned around the outer surface of the support structure;
an external channel on the outer surface of the support structure; and
an actuation line positioned at least in part in the external channel, the actuation line being in communication with the one or more ligation bands such that actuation of the actuation line deploys the one or more ligation bands.

10. The ligation banding cap assembly of claim 9, wherein the ligation banding cap assembly is adapted to be attached to the distal end of an elongated medical device.

11. The ligation banding cap assembly of claim 10, wherein the elongated medical device is an endoscope.

12. The ligation banding cap assembly of claim 9, wherein the ligation banding cap assembly comprises a first raised ridge on one side of the external channel and a second raised ridge on the other side of the external channel.

13. The ligation banding cap assembly of claim 9, wherein the ligation banding cap assembly is in the form of a recess in the outer surface of the support structure.

14. The ligation banding cap assembly of claim 9, wherein the support structure comprises a plurality of slots in a distal end, wherein the actuation line extends through the external channel, through at least two slots, and back along the outer surface of the support structure to the one or more ligation bands.

15. The ligation banding cap assembly of claim 9, wherein the actuation line comprises one or more enlarged structures for advancing the one or more ligation bands.

16. A method of assembling a ligation banding cap assembly to an elongated medical device, the method comprising:
positioning the ligation banding cap assembly relative to the elongated medical device, the ligation banding cap assembly comprising:
a support structure having a support structure lumen therethrough, the support structure having an outer surface;
one or more ligation bands positioned around the outer surface of the support structure;
an external channel on the outer surface of the support structure; and
an actuation line positioned at least in part in the external channel, the actuation line being in communication with the one or more ligation bands such that actuation of the actuation line deploys the one or more ligation bands; and
attaching the ligation banding cap assembly to the elongated medical device, with the actuation line external to the support structure lumen of the ligation banding cap assembly and external to the elongated medical device.

17. The method of claim 16, wherein the elongated medical device is an endoscope.

18. The method of claim 16, wherein the ligation banding cap assembly comprises a first raised ridge on one side of the external channel and a second raised ridge on the other side of the external channel.

19. The method of claim 16, wherein the ligation banding cap assembly is in the form of a recess in the outer surface of the support structure.

20. The method of claim 16, wherein the actuation line comprises one or more enlarged structures for advancing the one or more ligation bands.
